(19) United States
(12) Patent Application Publication
(10) Pub. No.: US 2020/0005332 A1
(43) Pub. Date: Jan. 2, 2020

(54) SYSTEMS, DEVICES, AND METHODS FOR PROVIDING SUPPLY CHAIN AND ETHICAL SOURCING INFORMATION ON A PRODUCT

(71) Applicant: L'OREAL, Paris (FR)

(72) Inventor: Sandrine GADOL, New York, NY (US)

(73) Assignee: L'OREAL, Paris (FR)

(21) Appl. No.: 16/023,157

(22) Filed: Jun. 29, 2018

(51) Int. Cl. G06Q 30/00; G06Q 30/06; G06Q 50/28; H04L 9/06; G06K 7/10

(52) U.S. Cl. CPC G06Q 30/018; G06Q 30/0627; A61K 8/00; H04L 9/0637; G06K 7/10; G06Q 50/28

(57) ABSTRACT

A system is provided for authenticating an origin of a cosmetic product, including: processing circuitry configured to receive occurrence information of a handling operation from each point in a manufacturing and distribution chain of the cosmetic product; output a result of a block chain operation to signify that each predetermined point in the manufacturing and distribution chain of the cosmetic product has provided occurrence information of a handling operation; receive code information, from a packaging location in the manufacturing and distribution chain, which corresponds to code information applied to a packaging of the cosmetic product, store the code information received from the packaging location in association with the block chain result; receive information from a user device inquiring about the cosmetic product; and output information about the cosmetic product to the mobile user device in response to the received information.

### BACKGROUND

FIG. 1 shows a conventional distribution chain 100 which involves a complex network of entities, such as a chemical provider, farmers, research centers, exporters, cooperatives, transporters, manufacturer processors, input suppliers, importers, wholesalers, retailers, and ultimately the consumer.

In view of increased reports of fraud and questions on the origins of source elements in various consumer products, reputable companies are seeking new solutions that allow them to easily trace and authenticate their products. Opaque supply chains present significant financial and reputational risks and with growing consumer demand for product transparency, it is now more important than ever for brands to be able to provide traceability on the origins of a product.

### BRIEF SUMMARY

According to an embodiment, a system is provided for authenticating an origin of a cosmetic product comprising: processing circuitry configured to: receive occurrence information of a handling operation from each point in a manufacturing and distribution chain of the cosmetic product; output a result of a block chain operation to signify that each predetermined point in the manufacturing and distribution chain of the cosmetic product has provided occurrence information of a handling operation; receive code information, from a packaging location in the manufacturing and distribution chain, which corresponds to code information applied to a packaging of the cosmetic product, store the code information received from the packaging location in association with the block chain result; receive information from a user device inquiring about the cosmetic product; and output information about the cosmetic product to the mobile user device in response to the received information.

According to an embodiment, the user device is a mobile device, and the processing circuitry is configured to receive code information from the mobile user device corresponding to code information scanned detected by the mobile device from a cosmetic product package at a retail location; determine if the received code information from the mobile user device corresponds to stored code information received from the packaging location that is stored with the block chain results; and output information about the cosmetic product to the mobile user device based on the determination result.

According to an embodiment, the information about the cosmetic product verifies an origin of one or more ingredients included in the cosmetic product.

According to an embodiment, the information about the cosmetic product verifies that the cosmetic product has followed an expected manufacturing and distribution chain to the retail location.

According to an embodiment, the information about the cosmetic product verifies that the cosmetic product has been made at facilities that follow ethical practices.

According to an embodiment, the information about the cosmetic product indicates one or more standard certifications attributable to the cosmetic product.

According to an embodiment, the information about the cosmetic product indicates a sustainability score of the cosmetic product.

According to an embodiment, the occurrence information of the handling operation is one of a receiving, storing, dispensing, or mixing operation related to an ingredient of the cosmetic product.

According to an embodiment, the code information applied to the packaging of the cosmetic product corresponds to a printed unique code or near field communication (NFC) information.

According to an embodiment, a method is provided, implemented by a system for authenticating an origin of a cosmetic product comprising: receiving occurrence information of a handling operation from each point in a manufacturing and distribution chain of the cosmetic product; outputting a result of a block chain operation to signify that each predetermined point in the manufacturing and distribution chain of the cosmetic product has provided occurrence information of a handling operation; receiving code information, from a packaging location in the manufacturing and distribution chain, which corresponds to code information applied to a packaging of the cosmetic product, store the code information received from the packaging location in association with the block chain result; receiving information from a user device inquiring about the cosmetic product; and outputting information about the cosmetic product to the mobile user device in response to the received information.

In the embodiments, the system provides transparency about not only the composition and manufacturing of the product but also the sourcing/origin of the product and any ingredients contained therein.

Furthermore, in an embodiment, the system comprises a system for authenticating an origin of one or more components, ingredients, elements, sources, venders, manufactures, distributors, and the like associated with one or more personal care products (e.g., personal hygiene products, cosmetic products, consumer products, skincare products, healthcare products, haircare products, nail care product, fragrances, toiletries, shaving products, etc., and the like.)

Further non-limiting examples of personal care products include body oils, body wash, cleansing pads, colognes, conditioners, cotton pads, cotton swabs, deodorants, eye liners, facial cleansers, facial tissues, facial treatments, fragrances, hair clippers, hand soaps, lip balms, lip glosses, lipsticks, lotion, makeup, makeup removers, moisturizers, nail files, perfumes, pomades, razors, shampoos, shaving creams, sunscreens, talcum powder, toilet paper, toothpaste, treatment masks, wet wipes, etc., and the like.

In an embodiment, the system improves how personal care product and their components are traced and authenticated to combat the problems associated with opaque supply chains. For example, in an embodiment, a system for authenticating an origin information associated with a personal care product includes a component configured receive occurrence information of a handling operation from each point in a sourcing, manufacturing, distribution chain, and the like of a personal care product.

### BRIEF DESCRIPTION OF THE DRAWINGS

FIG. 1 is a background art description of a conventional distribution chain 100.

FIG. 2 is a system of a manufacturing and distribution chain according to an embodiment

FIG. 3 is blockchain-based identity and transaction platform according to an embodiment.

FIGS. 4A, 4B, and 4C depict a user operation at a retail location according to an embodiment.

FIG. 5 shows a process performed at an individual source location according to an embodiment.

FIG. 6 shows a specific process performed by the Central Facility according to an embodiment.

FIG. 7 shows a blockchain process performed by the authentication management server according to an embodiment.

FIG. 8A shows a process performed by the mobile device according to an embodiment.

FIG. 8B shows an alternative process performed at an e-commerce website according to an embodiment

FIG. 9 shows a process performed by the authentication management center in response to receiving the scanned/detected code information from the mobile device according to an embodiment.

FIG. 10 is a detailed block diagram illustrating an exemplary user device according to an embodiment.

FIG. 11 shows an example of a device that may be implemented at any of the distribution locations and the authentication management center according to an embodiment.

FIG. 12 shows an example in which the user device is a voice-activated assistant according to an embodiment.

### DETAILED DESCRIPTION

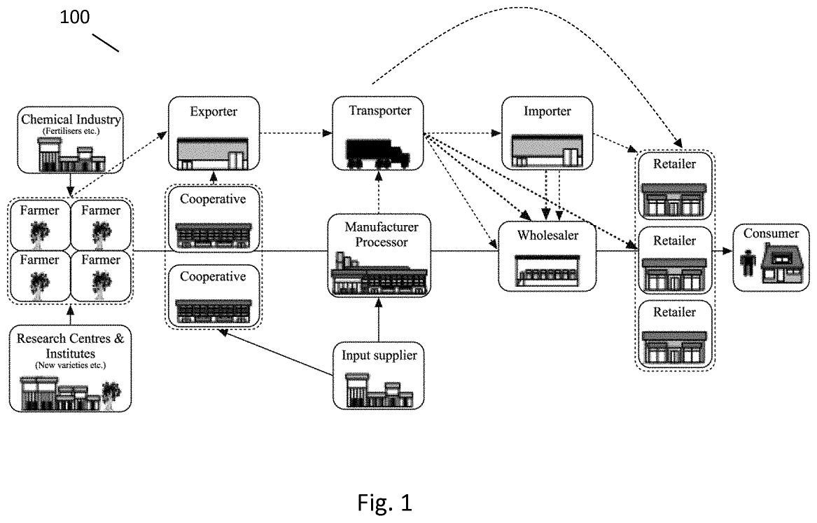

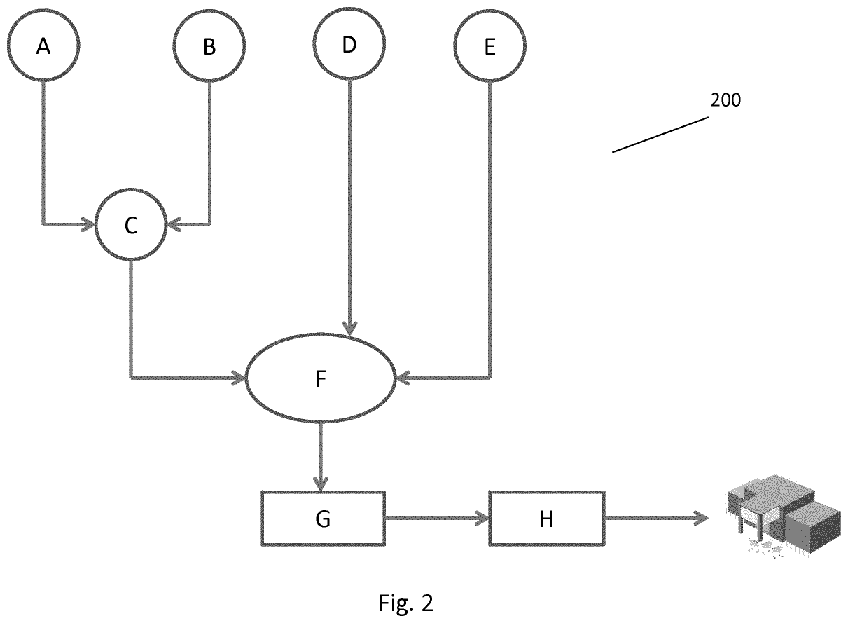

FIG. 2 shows an overview of a system 200, similar to that shown in FIG. 1 but simplified to provide a better understanding of the present embodiments. The system 200 implements a technique allowing verification and traceability, and full transparency on the origins of a cosmetic product, which may include a combination of various ingredients and/or chemicals in its composition. More specifically, the system allows transparency to the customer on the formula, packaging, manufacturing and transportation related to the cosmetic product. The system allows verification of the origin and safety of the materials or ingredients used. Such verification may confirm a standard certification (such as USDA organic), or it may provide a trusted sustainability score. Verification may also be provided to ensure ethical sourcing and production practices.

The system 200 includes chemical source locations A, B, C, D, and E. The chemical sources may be plants or processing facilities which provide one or more chemicals to be used as an ingredient in a cosmetic product. The chemicals may be directly provided to a central facility F, which combines the ingredients into the final product. Alternatively, the chemicals may be pre-combined or processed in certain instances prior to being provided to the central facility F, such as is the case for source chemicals from A and B being combined downstream at location C prior to being sent to central facility F.

The final product may be completed and packaged at the central facility F. Alternatively, any processes directed to completing the manufacture and packaging of the product may take place at separate locations as necessary, such as a central facility receiving turnkey products or products which are manufactured by subcontractors of outside partners, but they are depicted as occurring at central facility F to simplify the present analysis.

Following packaging at the central facility F, the final product may be distributed and transported at one or more distribution or transportation centers, depicted as G and H, before being received at a retailer.

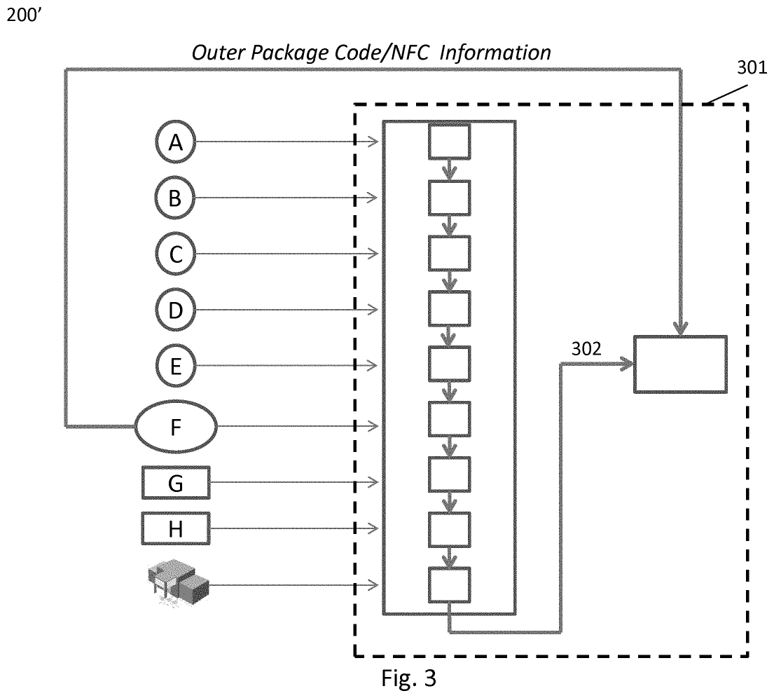

FIG. 3 shows a different view of the system 200 depicted in FIG. 2, but in a form shown as 200′ in which in which a blockchain-based identity and transaction platform can be implemented. In this system, each point in the distribution chain 200 sends data to an authentication management center 301. A “blockchain” is formed at the authentication management center 301.

As used herein, “blockchain” refers to a distributed storage platform and network in which individual “blocks” are connected in a chain. Each block is linked to the previous block in the blockchain by, for example, including a hash of the previous block as a “proof of work.” Various hash functions, including functions in the Secure Hash Algorithm (SHA)-1 or -2 families, such as SHA-256, can be used to perform a one-way hash. For a one-way hash, it is generally considered to be impossible or impractical to generate the input (the “message”) to the hash function based on the output (the “message digest” or “digest”) of the hash function.

In a blockchain, the individual blocks can store a variety of data that may or may not be related (e.g., may or may not be associated with a same user). In this case, the data sent by each point in the distribution chain represents completion of a step in the process of forming the cosmetic product and delivering the product to a point of retail.

For instance, at the chemical source locations, the following information may be sent to the authentication management center 301: inbound ingredient date/time; batch or lot number (when applicable); processing date/time (when applicable); outbound delivery date/time. In other words, every instance at which the ingredients that go towards the cosmetic product are handled, and every instance at which the completed cosmetic product itself is handled, is transmitted to the authentication management center 301. A one-way hash described above can be executed for each piece of data according to its expected sequence in the distribution chain to generate an output message 302.

Additionally, central facility F generates a unique code or near field communication (NFC) information which will be included on the packaging of the cosmetic product, and this code or NFC information will be sent to the authentication management center 301 to be associated with the output message 302.

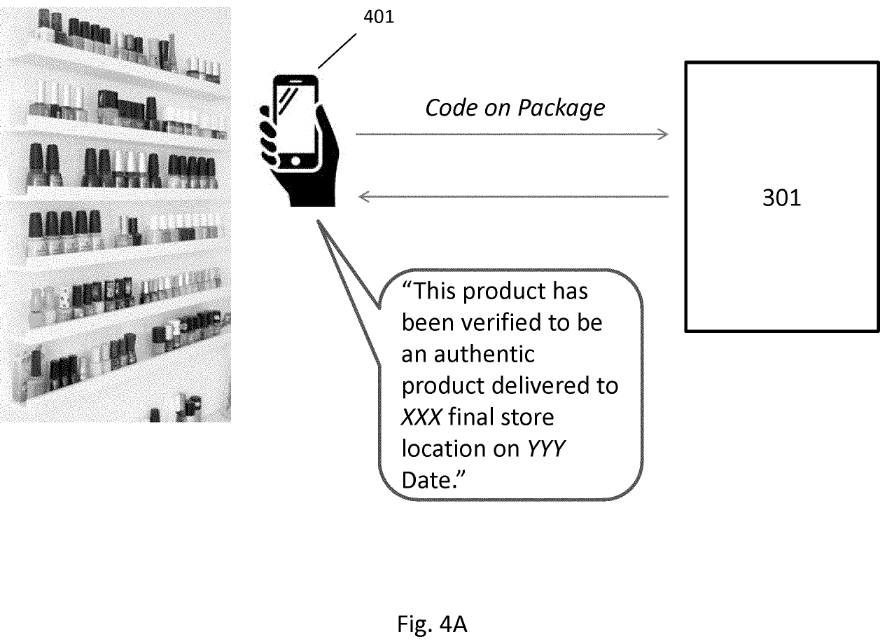

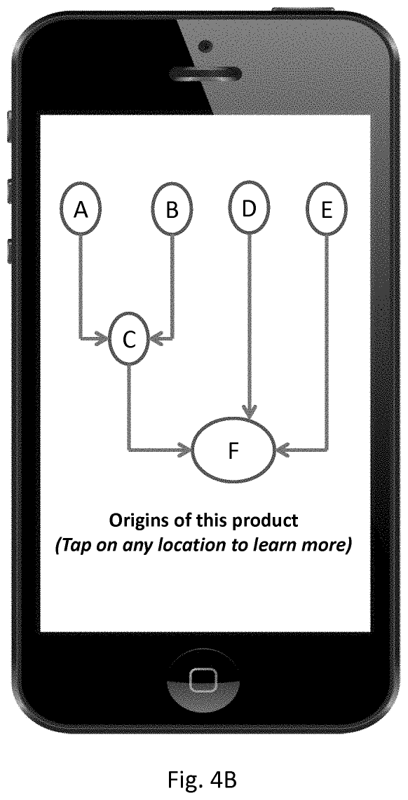

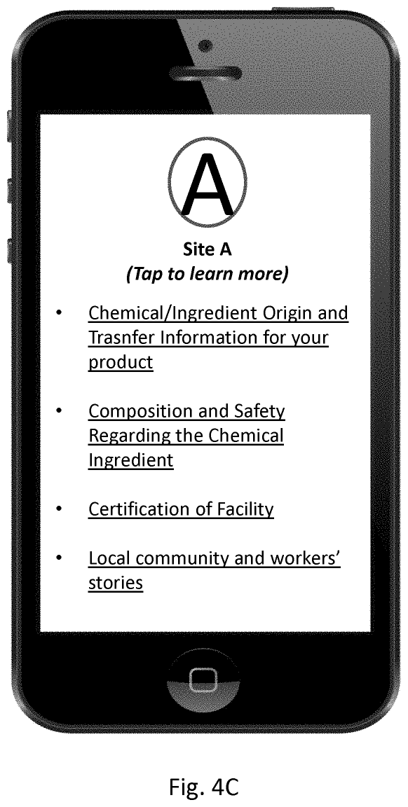

FIG. 4 depicts a user operation at a retail location according to an embodiment. In the embodiment, the user is operating a mobile device 401 (such as a “smartphone”). However, any other type of mobile device may be used, such as a laptop computer, a tablet device, or the like. The user, having physical access to the cosmetic product, scans or detects the unique which has been applied to the cosmetic product package at the central facility F. The manner in which the user scans or detects the code on the product package may vary depending on the type of code which is applied. If the code is stored on an NFC radio tag applied to the package, then the user may initiate reading of the data on the NFC (or RFID) radio tag using an NFC reader included in the mobile device 401. Such methods of utilizing an NFC reader to read data off of an NFC radio tag are well understood in the art.

Alternatively, the product package may include a visible printed code such as a bar code or a QR code. The user may use an image capturing device or other type of scanner to read the printed code and capture the unique identification data from the printed code. After detecting or reading the code on the package, the mobile device 401 transmits the code to the authentication management center 301.

As noted in the FIG. 3, the code 303 is associated with the output 302 of the blockchain, which as discussed above, may be the result of a one-way hashing algorithm over each piece of data provided from the individual locations in the distribution chain. As a result of the blockchain being completed all through to the data being received from the final retail location acting as the final destination of the cosmetic product, the authentication management center may verify that the cosmetic product has properly reached the correct final destination.

With such verification complete, the authentication management center 301 may transmit information back to the mobile device 401 which allows the user to confirm that the product they are holding is the same product that has followed the correct distribution chain. For instance, as shown in FIG. 4, the mobile device 401 may display a message simply asking the user to confirm that the product they are holding is at their current location by indicating the store and date of the final product delivery. If this information is not correct, then the user will be aware that the product has not followed the correct distribution chain and therefore may not be trustworthy.

The actions performed user mobile device may be performed through a single application program that is stored on the user mobile device. Such an application may be provided by a company associated with the cosmetic product. Otherwise, the application may be provided a company associated with the authentication management center, especially if such company is considered to be independent of the cosmetic product company to provide additional trust in the independence of the verification process.

Besides the example described above, as illustrated in FIGS. 4B and 4C, additional information may extracted and transmitted to the user from the authentication management center after the code is received as follows.
Origin and transfer information of each separate chemical or ingredient used to make the cosmetic product. This information can be verified based on a lot or batch number that is scanned from a container that holds the chemical or ingredient at any point in the distribution process. This information can also be verified based on a dispensing device that is used to dispense the chemical from one point to another at any point in the manufacturing process. In other words, while it may be unfeasible to literally track the actual chemical, it is feasible to track the physical containers or devices which handle the chemical and to further track each storage, dispensing, or mixing operation related to each chemical.
Information on the composition and safety of each separate chemical or ingredient used to make the cosmetic product. This is information on the actual chemical or ingredient itself. This may include safety information such as hazards, side effects, and allergy information.
Background information on facilities and/or location responsible for handling each separate chemical or ingredient used to make the cosmetic product. This information is related to reputational information on the specific locations, such as recent news, certifications and inspection information, ethical issues such as labor practices and/or animal testing involved with the specific location, and/or stories of the local workers at each location.

Accordingly, the above-described system provides transparency about not only the composition and manufacturing of the product but also the sourcing/origin of the product and any ingredients contained therein.

For instance, as shown in FIG. 4B, a user may select a particular distribution location in the distribution chain. If, for example, Site A is chosen, then a display may appear as shown in FIG. 4C, which provides several options, such as learning more about the chemical/ingredient used in the specific product in addition to the information about the facility at the distribution location itself.

All of the above information may be collected ahead of time and associated with the source locations at the authentication management center. Alternatively, the above-noted information, or updates to the above-noted information may be sent along with the data that is transmitted from each location to the authentication management center as a chemical or ingredient makes its way through the distribution chain.

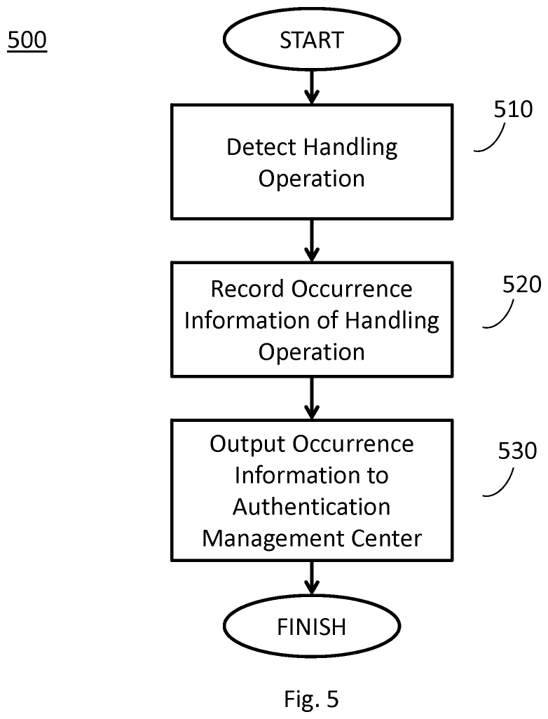

FIG. 5 shows a process 500 performed at an individual source location according to an embodiment. The process may be executed by a computing device located at the source location, which is further connected to individual sensors or scanners (which may be operated automatically or manually operated). More information on the hardware and equipment utilized at each location will be described in further detail below.

In step 510, a “handling operation” will be detected at the location. The handling operation may be action taken with respect to a particular chemical ingredient, such as a receiving, storing, dispensing, or mixing operation related to each chemical. In step 520, information related to the detected handling operation will be recorded in a memory. The information stored may be information of a specified action which may further include amount, time, and date information (“5 g of tracked chemical XX dispensed from container A to mixing apparatus B at time HH:MM SS on Date MM/DD/YY”). In this specific example, the handling operation information may be transmitted by one device in the system (such as a mixing/dispensing apparatus) and transmitted to the computing device at the source location for storing in memory. When the handling operation involves transport of a chemical from one location to another, the handling operation may be detected by scanning the container at the time of reception at each destination.

By detecting such handling operations, specific quantities of chemicals can be tracked as they move through an entire process depending on the level of granularity required. For instance, if a chemical is purchased from a supplier, the tracking may be performed at the point of receiving the purchased chemical. Otherwise, the tracking may go all the way to point of chemical creation or even to the process of extracting or mining elements from natural sources.

In step 530, the occurrence information of the handling operation is transmitted from the computing unit at the source location to the authentication management center as is depicted in FIG. 3.

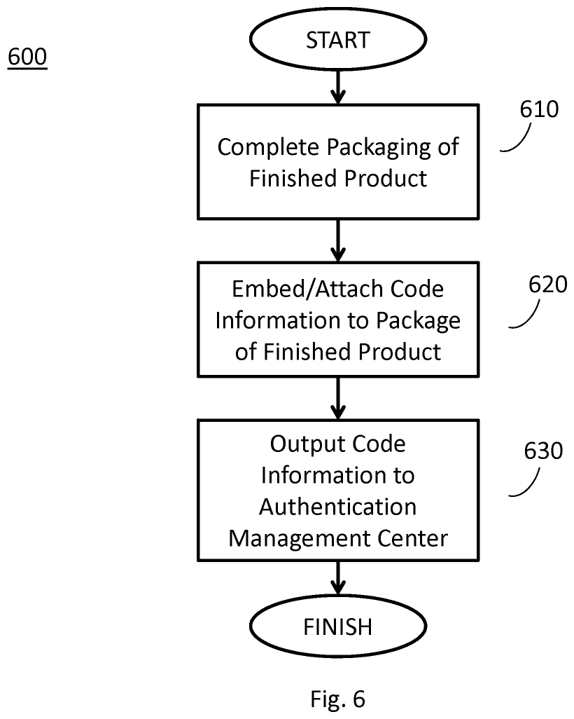

FIG. 6 shows a specific process 600 performed by the Central Facility F. As noted above, in FIG. 3, the Central Facility F is responsible for packaging the completed product (step 610). After packaging the completed product, the central facility adds code information to the package (step 620). As described above, the code may be stored in NFC radio tag that is physically applied to the package according to methods understood in the art. Alternatively, the Central Facility F may add a visible printed code such as a bar code or a QR code to the package according to methods understood in the art. After the code is added, the data in the code is transmitted to the authentication management center (such as over a network connection) (step 630).

It is noted that while Central Facility F performs the process 600, Central Facility F also performs the process 500 shown in FIG. 5 to contribute to the blockchain.

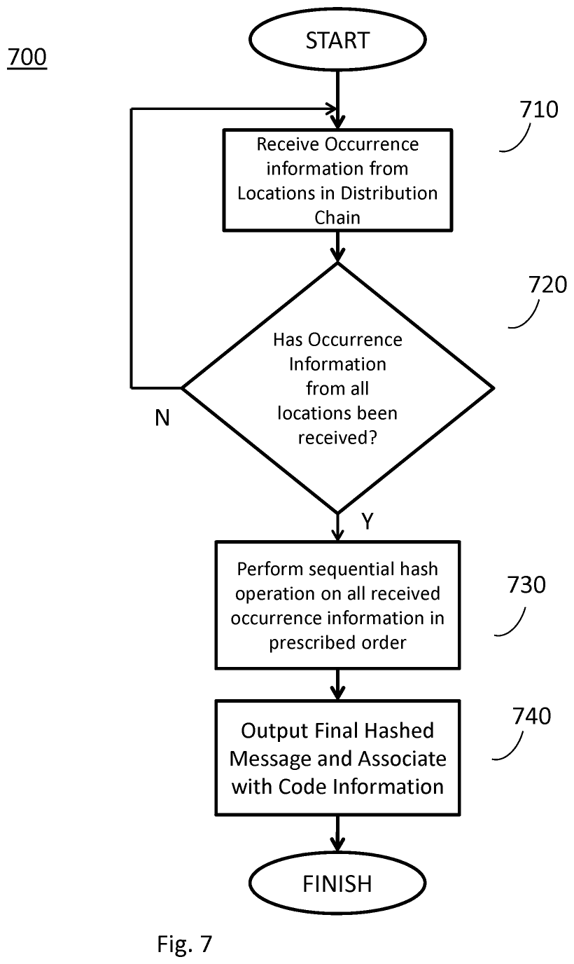

FIG. 7 shows a blockchain process 700 performed by the authentication management server 301. At step 710, the authentication management server receives the occurrence information of the handling operations from each location after each location performs the process 500 shown in FIG. 5. After the occurrence information from all of the locations has properly been received (step 720), the authentication management center performs a blockchain hashing operation on all of the received data in a prescribed order. The prescribed order may be provided by the product manufacturer(s) in advance. The final step in the blockchain will result in a final hashed message that is associated with the code information received from the central facility F. to the end the process (740).

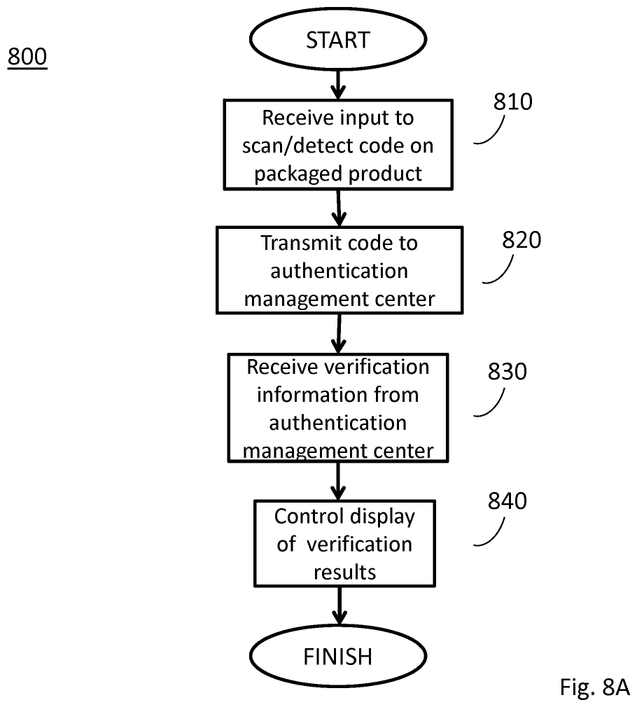

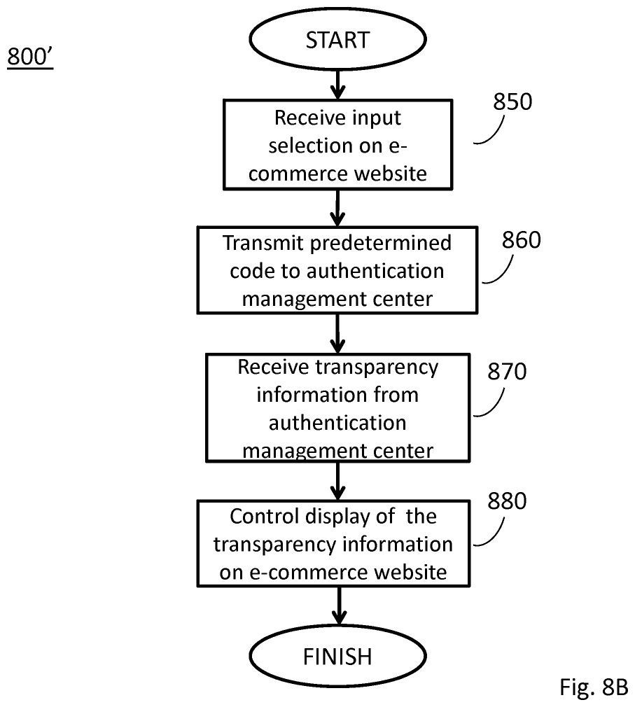

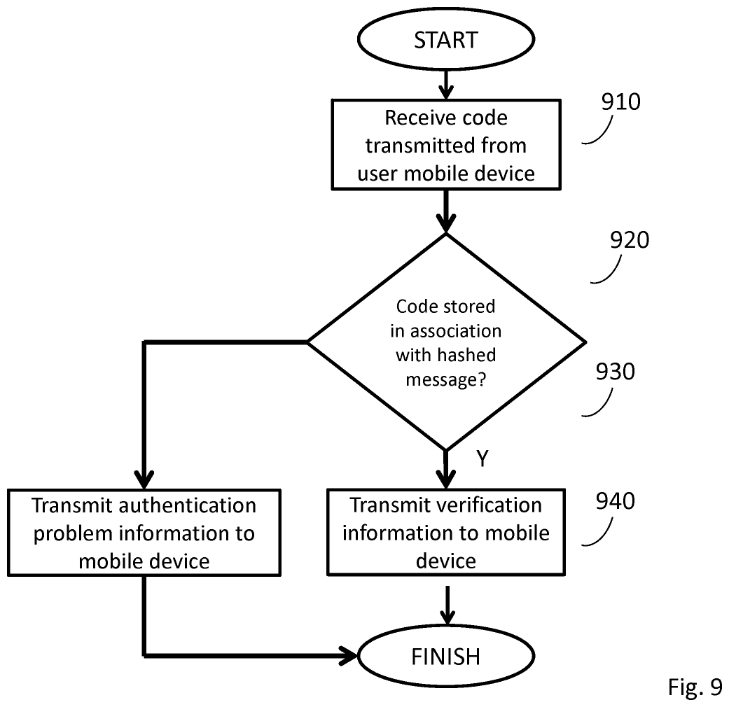

FIG. 8A shows a process performed by the mobile device 401. At step 810, the user will provide an input to scan or detect the code on a packaged product that this physically accessible at a retail center. The user may be within an application before providing the input, and the input may be in the form of touching a graphic button displayed on the mobile device, but it is not limited to such. After the code is successfully obtained, the code is transmitted to an authentication management center via a network, such as the Internet (step 820). The mobile device will wait and receive a response from the authentication management center (based on a process shown in FIG. 9) in step 830. The mobile device will then be controlled to the display the results of the verification process. One example of the displayed result will be the message shown in FIG. 4 in which the message displays the correct store location and delivery date for the user. A further step may be provided (not shown) in which the user is prompted via the display to confirm the displayed information is correct.

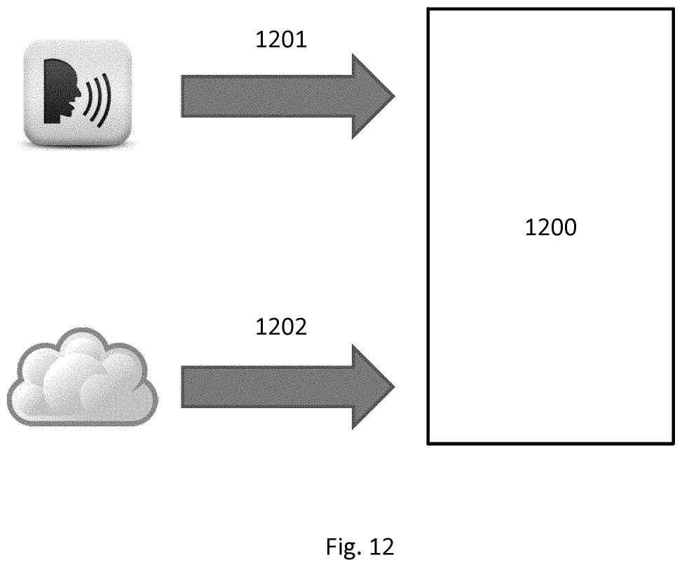

FIG. 8B shows an alternative process 801′ performed when the user is purchasing a product from an e-commerce website in which the in-person scan of a code on the packaged product is not available. In step 850, the user will provide an input of a selection of a learning the general transparency information of the product, such as the expected origin information, certifications, and/or distribution chain which is pre-stored for the product. Such selection may be provided by the click of a graphic button on the user's computer or mobile device. Alternatively, with respect to the embodiment shown in FIG. 12 and described below, if the user is operating a voice-activated assistant, the user may request an audible description of the transparency information using a voice command, such as “please tell me the source of the main ingredients in [product name].” The types of voice commands are flexible as is understood in the art. The e-commerce website provider, then may transmit a predetermined code (such as the serial number of the product) to the authentication management center at step 860. The website provider then receives the above-described transparency information from the authentication management center at step 870. The website provider then controls display of the transparency information on the website. Alternatively, the website provider may receive the transparency information ahead of time for any product that is being sold on the website, so the information may be displayed immediately for the user instead of requiring transmission of the predetermined code to the authentication management center.

FIG. 9 shows a process performed by the authentication management center in response to receiving the scanned/detected code information from the mobile device 401. At step 910, the authentication management center receives the scanned/detected code information from the mobile device 401. At step 920, the authentication management center looks up stored code information to determine if the received code information matches code information that has been received from the central facility F which is associated with a final hashed message resulting from the completion of the block chain. If there is a match, then the process proceeds to step 940, at which the verification information describe above may be transmitted to the mobile device to cause displayed result to be displayed on the mobile device. If there is not match, then there is a problem in the distribution chain and the code on the product package at the user mobile device cannot be authenticated as corresponding to a product package that has been delivered to its intended final destination. In that case, the authentication management center transmits authentication problem information to the mobile device at step 940, which will cause a display message to be displayed at the mobile device intended to alert the user of a problem. The user may be prompted to bring the product package to the manager of the retail store and/or to select another product package.

It is noted that the above-described embodiments provide a clear improvement to the technological environment of electronic tracking of the source/origin of a product for a user. The embodiments provide improvements in tracing and authenticating an origin of one or more components, ingredients, elements, venders, manufactures, distributors, and the like associated with a personal care product.) In at least one particular case, the implementation of the end-to-end blockchain, the results of which are accessible to the user through the user device at the point of sale, allow much more transparency on a wide variety of characteristics related to the product, and even alert the user to a potential authenticity problem with the product, without requiring manual human oversight or research.

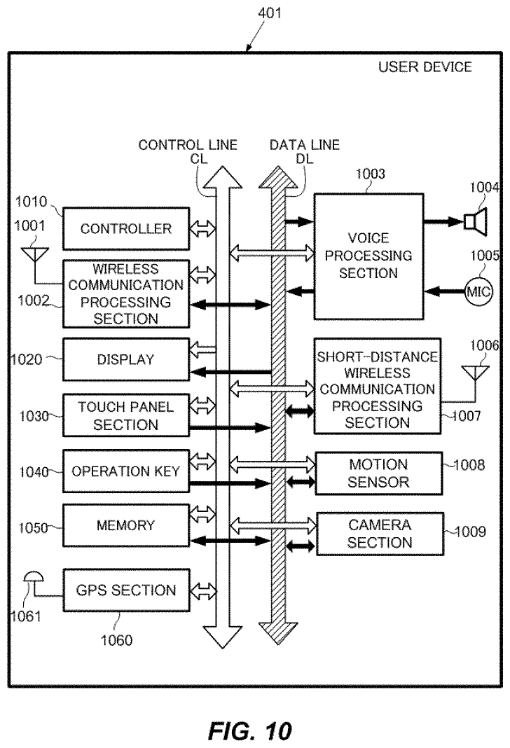

FIG. 10 is a more detailed block diagram illustrating an exemplary user device 401 according to certain embodiments of the present disclosure. In certain embodiments, user device 401 may be a smartphone. However, the skilled artisan will appreciate that the features described herein may be adapted to be implemented on other devices (e.g., a laptop, a tablet, a server, an e-reader, a camera, a navigation device, etc.). The exemplary user device 401 of FIG. 10 includes a controller 1010 and a wireless communication processor 1002 connected to an antenna 1001. A speaker 1004 and a microphone 1005 are connected to a voice processor 1003.

The controller 1010 may include circuitry such as one or more Central Processing Units (CPUs), and may control each element in the user device 401 to perform functions related to communication control, audio signal processing, control for the audio signal processing, still and moving image processing and control, and other kinds of signal processing. The controller 1010 may perform these functions by executing instructions stored in a memory 1050. Alternatively or in addition to the local storage of the memory 150, the functions may be executed using instructions stored on an external device accessed on a network or on a non-transitory computer readable medium.

The memory 1050 includes but is not limited to Read Only Memory (ROM), Random Access Memory (RAM), or a memory array including a combination of volatile and non-volatile memory units. The memory 1050 may be utilized as working memory by the controller 1010 while executing the processes and algorithms of the present disclosure. Additionally, the memory 1050 may be used for long-term storage, e.g., of image data and information related thereto. As disclosed in relation to FIG. 1, the memory 1050 may be configured to store the battle view information, operation view information and list of commands.

The user device 401 includes a control line CL and data line DL as internal communication bus lines. Control data to/from the controller 1010 may be transmitted through the control line CL. The data line DL may be used for transmission of voice data, display data, etc.

The antenna 1001 transmits/receives electromagnetic wave signals between base stations for performing radio-based communication, such as the various forms of cellular telephone communication. The wireless communication processor 1002 controls the communication performed between the user device 401 and other external devices via the antenna 1001. For example, the wireless communication processor 1002 may control communication between base stations for cellular phone communication.

The speaker 1004 emits an audio signal corresponding to audio data supplied from the voice processor 1003. The microphone 1005 detects surrounding audio and converts the detected audio into an audio signal. The audio signal may then be output to the voice processor 1003 for further processing. The voice processor 1003 demodulates and/or decodes the audio data read from the memory 1050 or audio data received by the wireless communication processor 102 and/or a short-distance wireless communication processor 1007. Additionally, the voice processor 1003 may decode audio signals obtained by the microphone 1005.

The exemplary user device 401 may also include a display 1020, a touch panel 1030, an operation key 1040, and a short-distance communication processor 1007 connected to an antenna 1006. The display 1020 may be a Liquid Crystal Display (LCD), an organic electroluminescence display panel, or another display screen technology. In addition to displaying still and moving image data, the display 1020 may display operational inputs, such as numbers or icons which may be used for control of the user device 401. The display 1020 may additionally display a GUI for a user to control aspects of the user device 401 and/or other devices. Further, the display 1020 may display characters and images received by the user device 401 and/or stored in the memory 1050 or accessed from an external device on a network. For example, the user device 401 may access a network such as the Internet and display text and/or images transmitted from a Web server.

The touch panel 1030 may include a physical touch panel display screen and a touch panel driver. The touch panel 1030 may include one or more touch sensors for detecting an input operation on an operation surface of the touch panel display screen. The touch panel 1030 also detects a touch shape and a touch area. Used herein, the phrase “touch operation” refers to an input operation performed by touching an operation surface of the touch panel display with an instruction object, such as a finger, thumb, or stylus-type instrument. In the case where a stylus or the like is used in a touch operation, the stylus may include a conductive material at least at the tip of the stylus such that the sensors included in the touch panel 1030 may detect when the stylus approaches/contacts the operation surface of the touch panel display (similar to the case in which a finger is used for the touch operation).

One or more of the display 1020 and the touch panel 1030 are examples of the touch panel display 25 depicted in FIG. 10 and described above.

In certain aspects of the present disclosure, the touch panel 1030 may be disposed adjacent to the display 1020 (e.g., laminated) or may be formed integrally with the display 1020. For simplicity, the present disclosure assumes the touch panel 130 is formed integrally with the display 1020 and therefore, examples discussed herein may describe touch operations being performed on the surface of the display 1020 rather than the touch panel 1030. However, the skilled artisan will appreciate that this is not limiting.

For simplicity, the present disclosure assumes the touch panel 1030 is a capacitance-type touch panel technology. However, it should be appreciated that aspects of the present disclosure may easily be applied to other touch panel types (e.g., resistance-type touch panels) with alternate structures. In certain aspects of the present disclosure, the touch panel 1030 may include transparent electrode touch sensors arranged in the X-Y direction on the surface of transparent sensor glass.

The touch panel driver may be included in the touch panel 130 for control processing related to the touch panel 1030, such as scanning control. For example, the touch panel driver may scan each sensor in an electrostatic capacitance transparent electrode pattern in the X-direction and Y-direction and detect the electrostatic capacitance value of each sensor to determine when a touch operation is performed. The touch panel driver may output a coordinate and corresponding electrostatic capacitance value for each sensor. The touch panel driver may also output a sensor identifier that may be mapped to a coordinate on the touch panel display screen. Additionally, the touch panel driver and touch panel sensors may detect when an instruction object, such as a finger is within a predetermined distance from an operation surface of the touch panel display screen. That is, the instruction object does not necessarily need to directly contact the operation surface of the touch panel display screen for touch sensors to detect the instruction object and perform processing described herein. For example, in certain embodiments, the touch panel 1030 may detect a position of a user's finger around an edge of the display panel 1020 (e.g., gripping a protective case that surrounds the display/touch panel). Signals may be transmitted by the touch panel driver, e.g. in response to a detection of a touch operation, in response to a query from another element based on timed data exchange, etc.

The touch panel 1030 and the display 1020 may be surrounded by a protective casing, which may also enclose the other elements included in the user device 401. In certain embodiments, a position of the user's fingers on the protective casing (but not directly on the surface of the display 1020) may be detected by the touch panel 130 sensors. Accordingly, the controller 1010 may perform display control processing described herein based on the detected position of the user's fingers gripping the casing. For example, an element in an interface may be moved to a new location within the interface (e.g., closer to one or more of the fingers) based on the detected finger position.

Further, in certain embodiments, the controller 1010 may be configured to detect which hand is holding the user device 401, based on the detected finger position. For example, the touch panel 1030 sensors may detect a plurality of fingers on the left side of the user device 401 (e.g., on an edge of the display 1020 or on the protective casing), and detect a single finger on the right side of the user device 401. In this exemplary scenario, the controller 1010 may determine that the user is holding the user device 401 with his/her right hand because the detected grip pattern corresponds to an expected pattern when the user device 401 is held only with the right hand.

The operation key 1040 may include one or more buttons or similar external control elements, which may generate an operation signal based on a detected input by the user. In addition to outputs from the touch panel 1030, these operation signals may be supplied to the controller 1010 for performing related processing and control. In certain aspects of the present disclosure, the processing and/or functions associated with external buttons and the like may be performed by the controller 1010 in response to an input operation on the touch panel 1030 display screen rather than the external button, key, etc. In this way, external buttons on the user device 401 may be eliminated in lieu of performing inputs via touch operations, thereby improving water-tightness.

The antenna 1006 may transmit/receive electromagnetic wave signals to/from other external apparatuses, and the short-distance wireless communication processor 1007 may control the wireless communication performed between the other external apparatuses. Bluetooth, IEEE 802.11, and near-field communication (NFC) are non-limiting examples of wireless communication protocols that may be used for inter-device communication via the short-distance wireless communication processor 1007.

The user device 401 may include a motion sensor 1008. The motion sensor 1008 may detect features of motion (i.e., one or more movements) of the user device 401. For example, the motion sensor 1008 may include an accelerometer to detect acceleration, a gyroscope to detect angular velocity, a geomagnetic sensor to detect direction, a geo-location sensor to detect location, etc., or a combination thereof to detect motion of the user device 401. In certain embodiments, the motion sensor 1008 may generate a detection signal that includes data representing the detected motion. For example, the motion sensor 1008 may determine a number of distinct movements in a motion (e.g., from start of the series of movements to the stop, within a predetermined time interval, etc.), a number of physical shocks on the user device 401 (e.g., a jarring, hitting, etc., of the electronic device), a speed and/or acceleration of the motion (instantaneous and/or temporal), or other motion features. The detected motion features may be included in the generated detection signal. The detection signal may be transmitted, e.g., to the controller 110, whereby further processing may be performed based on data included in the detection signal. The motion sensor 1008 can work in conjunction with a Global Positioning System (GPS) section 1060. The GPS section 1060 detects the present position of the terminal device 100. The information of the present position detected by the GPS section 1060 is transmitted to the controller 1010. An antenna 1061 is connected to the GPS section 1060 for receiving and transmitting signals to and from a GPS satellite.

The user device 401 may include a camera section 1009, which includes a lens and shutter for capturing photographs of the surroundings around the user device 401. In an embodiment, the camera section 1009 captures surroundings of an opposite side of the user device 401 from the user. The images of the captured photographs can be displayed on the display panel 1020. A memory section saves the captured photographs. The memory section may reside within the camera section 1009 or it may be part of the memory 1050. The camera section 1009 can be a separate feature attached to the user device 401 or it can be a built-in camera feature.

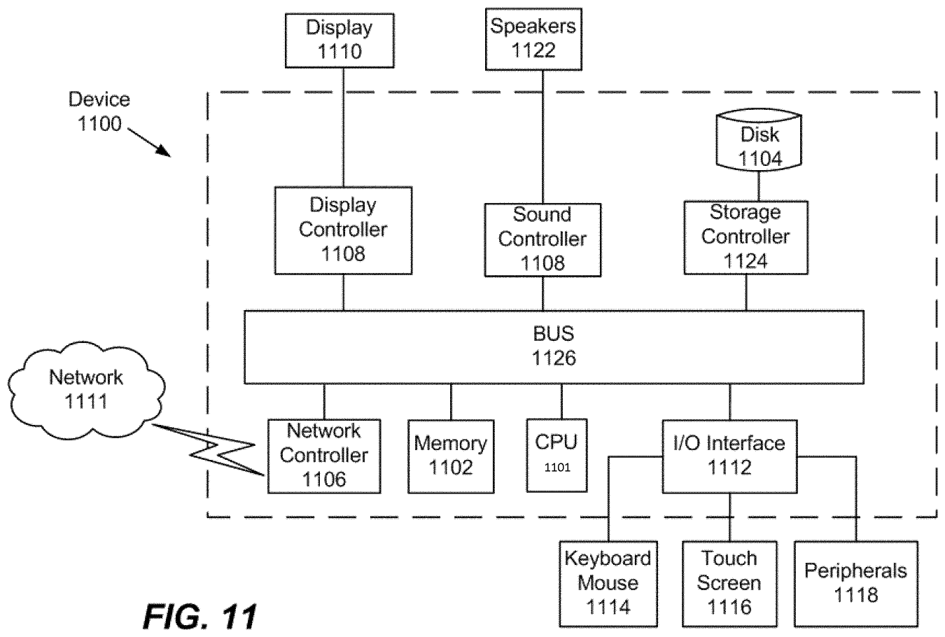

FIG. 11 shows an example of a device 1100 that may be implemented at any of the distribution locations and the authentication management center for performing any of the above-described functions.

In FIG. 11, the device 1100 includes a CPU 1101 which performs the processes described above/below. The process data and instructions may be stored in memory 1102. These processes and instructions may also be stored on a storage medium disk 1104 such as a hard drive (HDD) or portable storage medium or may be stored remotely. Further, the claimed advancements are not limited by the form of the computer-readable media on which the instructions of the inventive process are stored. For example, the instructions may be stored on CDs, DVDs, in FLASH memory, RAM, ROM, PROM, EPROM, EEPROM, hard disk or any other information processing device with which the device 1100 communicates, such as a server or computer.

Further, the claimed advancements may be provided as a utility application, background daemon, or component of an operating system, or combination thereof, executing in conjunction with CPU 1100 and an operating system such as Microsoft Windows 7, UNIX, Solaris, LINUX, Apple MAC-OS and other systems known to those skilled in the art. The hardware elements in order to achieve the device 1100 may be realized by various circuitry elements, known to those skilled in the art. For example, CPU 1100 may be a Xenon or Core processor from Intel of America or an Opteron processor from AMD of America, or may be other processor types that would be recognized by one of ordinary skill in the art. Alternatively, the CPU 1100 may be implemented on an FPGA, ASIC, PLD or using discrete logic circuits, as one of ordinary skill in the art would recognize. Further, CPU 1101 may be implemented as multiple processors cooperatively working in parallel to perform the instructions of the inventive processes described above.

The device 1100 in FIG. 11 also includes a network controller 1106, such as an Intel Ethernet PRO network interface card from Intel Corporation of America, for interfacing with network 1111. As can be appreciated, the network 1111 can be a public network, such as the Internet, or a private network such as an LAN or WAN network, or any combination thereof and can also include PSTN or ISDN sub-networks. The network 1111 can also be wired, such as an Ethernet network, or can be wireless such as a cellular network including EDGE, 3G and 4G wireless cellular systems. The wireless network can also be WiFi, Bluetooth, or any other wireless form of communication that is known.

The device 1100 further includes a display controller 1108, such as a NVIDIA GeForce GTX or Quadro graphics adaptor from NVIDIA Corporation of America for interfacing with display 1110, such as a Hewlett Packard HPL 2445w LCD monitor. A general purpose I/O interface 1112 interfaces with a keyboard and/or mouse 1114 as well as a touch screen panel 1116 on or separate from display 1110. General purpose I/O interface also connects to a variety of peripherals 1118 including printers and scanners, such as an OfficeJet or DeskJet from Hewlett Packard.

A sound controller 1120 is also provided in the device 1100, such as Sound Blaster X-Fi Titanium from Creative, to interface with speakers/microphone 1122 thereby providing sounds and/or music.

The general purpose storage controller 1124 connects the storage medium disk 1104 with communication bus 1126, which may be an ISA, EISA, VESA, PCI, or similar, for interconnecting all of the components of the device 1100. A general purpose I/O interface 1112 is omitted herein for brevity as these features are known.

FIG. 12 shows an alternative example in which the user device is a voice-activated assistant 1200. In general, there are two types of events which trigger action by the voice-activated as shown in FIG. 12. For instance, there are user-initiated actions 1201 and there are automated actions 1202 based on a predetermined schedule, history information, or trigger received over the network.

As an example of a user-initiated action 1201, the user may query the assistant 1200 for specific advice or a question about a product. In other words, in response to the user query, the assistant 1200 is able to provide a personalized response based on intelligently combining information taken from general available information that matches the user's needs. Therefore, in the present embodiment, the user may ask about a product, and also ask about transparency information regarding the product. While the user may not be able to scan or detect a code on a product or package, the user can receive general transparency about the product based on the expected origins and certifications of the product line.

The foregoing examples are illustrative of certain functionality of embodiments of the invention and are not intended to be limiting. Indeed, other functionality will be described below and other possible use cases will be apparent to the skilled artisan upon review of this disclosure.

What is claimed is:

1. A system for authenticating an origin of a cosmetic product comprising:
processing circuitry configured to:
receive occurrence information of a handling operation from each point in a manufacturing and distribution chain of the cosmetic product;
output a result of a block chain operation to signify that each predetermined point in the manufacturing and distribution chain of the cosmetic product has provided occurrence information of a handling operation;
receive code information, from a packaging location in the manufacturing and distribution chain, which corresponds to code information applied to a packaging of the cosmetic product, store the code information received from the packaging location in association with the block chain result;
receive information from a user device inquiring about the cosmetic product; and
output information about the cosmetic product to the mobile user device in response to the received information.

2. The system according to claim 1, wherein the user device is a mobile device, and the processing circuitry is configured to
receive code information from the mobile user device corresponding to code information scanned detected by the mobile device from a cosmetic product package at a retail location;
determine if the received code information from the mobile user device corresponds to stored code information received from the packaging location that is stored with the block chain results; and
output information about the cosmetic product to the mobile user device based on the determination result.

3. The system according to claim 1, wherein the information about the cosmetic product verifies an origin of one or more ingredients included in the cosmetic product.

4. The system according to claim 1, wherein the information about the cosmetic product verifies that the cosmetic product has followed an expected manufacturing and distribution chain to the retail location.

5. The system according to claim 1, wherein the information about the cosmetic product verifies that the cosmetic product has been made at facilities that follow ethical practices.

6. The system according to claim 1, wherein the information about the cosmetic product indicates one or more standard certifications attributable to the cosmetic product.

7. The system according to claim 1, wherein the information about the cosmetic product indicates a sustainability score of the cosmetic product.

8. The system according to claim 1, wherein the occurrence information of the handling operation is one of a receiving, storing, dispensing, or mixing operation related to an ingredient of the cosmetic product.

9. The system according to claim 1, wherein the code information applied to the packaging of the cosmetic product corresponds to a printed unique code or near field communication (NFC) information.

10. A method, implemented by a system for authenticating an origin of a cosmetic product comprising:
receiving occurrence information of a handling operation from each point in a manufacturing and distribution chain of the cosmetic product;
outputting a result of a block chain operation to signify that each predetermined point in the manufacturing and distribution chain of the cosmetic product has provided occurrence information of a handling operation;
receiving code information, from a packaging location in the manufacturing and distribution chain, which corresponds to code information applied to a packaging of the cosmetic product, store the code information received from the packaging location in association with the block chain result;
receiving information from a user device inquiring about the cosmetic product; and
outputting information about the cosmetic product to the mobile user device in response to the received information.

11. The method according to claim 10, wherein the user device is a mobile device, and the method includes
receiving code information from the mobile user device corresponding to code information scanned detected by the mobile device from a cosmetic product package at a retail location;
determining if the received code information from the mobile user device corresponds to stored code information received from the packaging location that is stored with the block chain results; and
outputting information about the cosmetic product to the mobile user device based on the determination result.

12. The method according to claim 10, wherein the information about the cosmetic product verifies an origin of one or more ingredients included in the cosmetic product.

13. The method according to claim 10, wherein the information about the cosmetic product verifies that the cosmetic product has followed an expected manufacturing and distribution chain to the retail location.

14. The method according to claim 10, wherein the information about the cosmetic product verifies that the cosmetic product has been made at facilities that follow ethical practices.

15. The method according to claim 10, wherein the information about the cosmetic product indicates one or more standard certifications attributable to the cosmetic product.

16. The method according to claim 10, wherein the information about the cosmetic product indicates a sustainability score of the cosmetic product.

17. The method according to claim 10, wherein the occurrence information of the handling operation is one of a receiving, storing, dispensing, or mixing operation related to an ingredient of the cosmetic product.

18. The method according to claim 10, wherein the code information applied to the packaging of the cosmetic product corresponds to a printed unique code or near field communication (NFC) information.
